(19) United States
(12) Patent Application Publication
(10) Pub. No.: US 2009/0123570 A1
(43) Pub. Date: May 14, 2009

(54) COMPOSITION AND METHOD FOR TREATING SORE THROAT

(76) Inventors: W. Randolph Warner, Punta Gorda, FL (US); Werner Gutmann, Powhatan, VA (US)

Correspondence Address: ELMORE PATENT LAW GROUP, PC, 515 Groton Road, Unit 1R, Westford, MA, 01886, US

(21) Appl. No.: 12/266,269

(22) Filed: Nov. 6, 2008

Related U.S. Application Data
(60) Provisional application No. 60/986,860, filed on Nov. 9, 2007.

(51) Int. Cl. A61K 33/14

(52) U.S. Cl. 424/677

(57) ABSTRACT

The present invention provides compositions and methods for treating sore throat by reducing inflammation and clearing oral-pharyngeal mucus associated with sore throat comprising hypertonic saline and menthol.

### RELATED APPLICATION

This application claims the benefit of U.S. Provisional Application No. 60/986,860, filed on Nov. 9, 2007. The entire teachings of the above application are incorporated herein by reference.

### BACKGROUND OF THE INVENTION

Sore throat, pharyngitis, laryngitis, excessive mucus and other mouth and throat irritations typically accompany common colds, influenza and like ailments and, as is well known, no cure or dependable preventative exists for such ailments. Apart from the mouth and throat irritations symptomatic of colds, inflammation of the throat tissues can also lead to more serious problems when accompanied by high grade fever, swollen glands, and difficult with breathing. Oral sore throat preparations, such as lozenges, sprays, solutions and the like, containing topical anesthetic/analgesic agents have long been used for the symptomatic relief of sore throat. For example, there are several over-the-counter oral throat sprays such as Chloraseptic® Throat Spray and Cepacol® Sore Throat Dual Relief Spray that use topical/local anesthetic(s) such as benzocaine, phenol and dyclonine as the active agents. However, these oral sore throat preparations aim at reducing pain and symptoms, and do not treat the underlying cause of sore throat which is inflammation.

Thus, the need still exists for an effective sore throat remedy that provides relief of symptoms, as well as reduces inflammation associated with sore throat.

### SUMMARY OF THE INVENTION

The present invention provides composition and method for treating sore throat by reducing inflammation and clearing oral-pharyngeal mucus associated with sore throat comprising hypertonic saline and menthol.

### DETAILED DESCRIPTION OF THE INVENTION

The discomfort associated with pharyngitis and generally referred to as a sore throat is the pain associated with inflammation of the oral pharynx. The pain results from the irritation of the exposed nerve endings located on the surface of the mucosa. The nerve endings are left exposed because the mucous barrier which is normally present in the oral pharynx has been disrupted. Pharyngitis is the result of various factors such as excessive smoking, alcohol, vapors or irritant chemicals and the onset of the malady of the common cold.

The present invention is based on the discovery that hypertonic saline reduces inflammation as well as clears oral-pharyngeal mucus associated with pharyngitis. The osmotic activity induced by the hypertonic saline reduces pressure caused by inflammation thereby relieving pain. The osmotic actions of the hypertonic saline also help clear excessive oral-pharyngeal mucus. When combined with at least menthol provides an effective sore throat remedy that soothes and relieves symptoms as well as reduces inflammation.

As used herein, the term “hypertonic” refers to a solution with a solute concentration that is higher than that inside cells present in that solution, and therefore causes water to diffuse out of the cells. The term “hypertonic” is a relational term expressing the greater relative solute concentration of one solution compared with another (i.e., the latter is “hypotonic” to the former). A hypertonic solution has a lower water potential than a solution that is hypotonic to it and has a correspondingly greater osmotic pressure.

As used herein, the term “saline” refers to salt, usually sodium chloride, but can include salt of potassium, magnesium or calcium. Physiological saline contains 20 0.9% of sodium chloride in water and is isotonic (i.e. having same osmotic pressure as blood serum).

As used herein, the term “osmotic activity” refers to the net diffusion of water across a selectively permeable membrane that is permeable in both directions to water, but varyingly permeable to solutes, wherein the water diffuses from one solution into another of lower water potential.

In one embodiment of the present invention provides therapeutic compositions for use in treating sore throat comprising menthol and an effective amount of hypertonic saline for osmotic action in the oral-pharyngeal epithelial mucosa thereby reduces pressure caused by inflammation.

In one embodiment of the present invention provides therapeutic compositions for use in treating sore throat comprising menthol and an effective amount of hypertonic saline that causes bacteria-cidal and bateria-static. In general, if an antibacterial agent is bacteriostatic, it means that the agent essentially stops bacterial cell growth (but does not kill the bacteria); if the agent is bacteriocidal, it means that the agent kills the bacterial cells (and may stop growth before killing the bacteria).

In one embodiment, the compositions of the invention comprise menthol and at least 1 percent of hypertonic saline, preferably between about 2 to about 30 percent of hypertonic saline, preferably between about 2 to about 20 percent of hypertonic saline, preferably between about 2 to about 15 percent of hypertonic saline, most preferably between about 3 to about 10 percent of hypertonic saline, most preferably between about 3 to about 7 percent of percent.

In one embodiment, the compositions of the invention comprise menthol and at about 3.5 percent of hypertonic saline.

In one embodiment, the compositions of the invention comprise menthol and at about 7 percent of hypertonic saline.

In one embodiment, the compositions of the invention comprise an effective amount of hypertonic saline and at least 0.02 percent menthol, preferably about 0.02 to about 15 percent menthol, preferably about 0.04 to about 10 percent menthol, most preferably about 0.04 to about 5 percent menthol.

In one embodiment, the compositions of the invention comprise menthol, effective amounts of hypertonic saline for osmotic actions in the oral-pharyngeal epithelial mucosa thereby reduces pressure caused by inflammation and are free of ascorbic acid.

A further object is to provide therapeutic compositions useful in alleviating the discomfort of pharyngitis in a human being suffering therefrom wherein said therapeutic compositions contain a therapeutically effective amount of a hypertonic saline and menthol.

A still further object is to provide therapeutic compositions useful in alleviating the discomfort of pharyngitis in a human being suffering therefrom wherein said therapeutic compositions contain about 0.5 to about 20 percent of a hypertonic saline and 0.02 to about 5 percent menthol.

In a further embodiment, the compositions for use in treating sore throat comprise concentration of about 3 to about 10 percent hypertonic saline, about 0.04 to about 5 percent menthol, and about 5 to about 20 percent benzocaine.

In a further embodiment, the compositions for use in treating sore throat comprise concentration of about 3 to about 10 percent hypertonic saline, about 0.04 to about 5 percent menthol, and about 0.05 to about 10 percent phenol.

In a further embodiment, the compositions for use in treating sore throat comprise concentration of about 3 to about 10 percent hypertonic saline, about 0.04 to about 5 percent menthol, and about 0.05 to about 10 percent dyclonine.

In a further embodiment, the compositions for use in treating sore throat comprise concentration of about 3 to about 10 percent hypertonic saline, about 0.04 to about 5 percent menthol, about 5 to about 20 percent benzocaine, and about 0.05 to about 10 percent phenol about 0.05 to about 10 percent dyclonine.

In one embodiment, any of the above compositions for the use in treating sore throat further comprise buffer agents, surfactants, preservatives, flavorings, topical oral anesthetics, anti-infectives, mucolytic agents, anti-inflammatory agents, analgesic herbs or combinations thereof.

Any surfactant is suitable, whether it is amphoteric, non-ionic, cationic or anionic. Examples of suitable surfactants include, but are not limited to, sodium lauryl sulfate, polysorbates such as polyoxyethylene sorbitan monooleate, monolaurate, monopalmitate, monstearate or another ester of polyoxyethylene sorbitan (e.g., the commercially available Tweens®, such as, Tween®20 and Tween®80 (ICI Speciality Chemicals)), sodium dioctylsulfosuccinate (DOSS), lecithin, stearylic alcohol, cetostearylic alcohol, cholesterol, polyoxyethylene ricin oil, polyoxyethylene fatty acid glycerides, poloxamers (e.g., Pluronics F68® and F108®, which are block copolymers of ethylene oxide and propylene oxide); polyoxyethylene castor oil derivatives; stearyltriethanolamine, laurylaminopropionic acid, lecithin, benzalkonium chloride, benzethonium chloride and glycerin monostearate; and hydrophilic polymers such as polyvinyl alcohol, polyvinylpyrrolidone, carboxymethylcellulose sodium, methylcellulose, hydroxymethylcellulose, hydroxyethylcellulose and hydroxypropylcellulose or mixtures thereof.

The preservatives include antimicrobial agents that kill and/or inhibit the proliferation and/or growth of microbes, particularly bacteria, fungi and yeast. Preservatives can be synthetic compounds, semisynthetic compounds, and naturally produced compounds. Suitable dermatologically absorbable preservatives include erythromycin, bacitracin, zinc bacitracin, polymycin, neomycin, chloramphenicol, tetracycline, sulfacetamide, minocycline, clindamycin, doxycycline, undecylenic acid and salts thereof, propionic acid and salts thereof, caprylic acid and salts thereof, ciprofloxacin, cephlasporins, benzoic acid, ciclopiroxolamine, clotrimazole, econazole nitrate, metronizadole, miconazole nitrate, ketacanazole, oxiconazole, tolnaftate, benzalkonium chloride, parabens, methyl paraben, benzethonium chloride, Neolone 950, sodium benzoate, sodium bisulfite, phenol, alkyl esters of parahydroxybenzoic acid, o-phenylphenol benzoic acid and salts thereof, boric acid and salts thereof, sorbic acid and salts thereof, chlorobutanol, benzyl alcohol, thimerosal, phenylmercuric acetate and nitrate, nitromersol, and cetylpyridinium chloride.

The term flavoring agent is meant to include flavors, cooling agents, sensates, and the like. The flavors used in the various layers of the confectionary product may include cooling agents such as menthol, as well as essential oils, synthetic flavors, or mixtures including but not limited to oils derived from plants and fruits such as citrus oils, fruit essences, peppermint oil, spearmint oil, clove oil, oil of wintergreen, anise and the like. The flavoring agents may include, but are not limited to, fruit flavor flavoring agents and savory flavor flavoring agents, as noted above. Suitable fruit flavor flavoring agents may include orange, lemon lime, grape, wild watermelon kiwi, cherry, pink lemonade, berry blue, strawberry, apple, plum, raisin, banana, pear, peach, figs, dates, and the like, and combinations thereof. Those skilled in the art will recognize that natural and artificial flavoring may be combined in any sensorially acceptable blend. All such flavors and flavor blends are contemplated by the present invention.

The term “anesthetic” refers to an agent that causes loss of sensation in a human or other mammal with or without the loss of consciousness. More particularly, the term “local anesthetic” refers to an anesthetic agent that induces local anesthesia by reversibly inhibiting peripheral nerve excitation and/or conduction. Local anesthetics suitable for use in the present invention include, but are not limited to, ester-based anesthetics, amide-based anesthetics, ester analogs of amide-based anesthetics, and ester analogs of other anesthetics. Ester-based anesthetics include, but are not limited to, cocaine, procaine, 2-chloroprocaine, tetracaine, benzocaine, amethocaine, chlorocaine, butamben, dibucaine, and the like. Amide-based anesthetics include, but are not limited to, lidocaine, prilocaine, mepivacaine, ropivocaine, etidocaine, levobupivacaine, bupivacaine, and the like. Other anesthetics suitable for use in the present invention include, but are not limited to, ester analogs of aconitine, dyclonine, ketamine, pramoxine, safrole, salicyl alcohol, and phenol.

The term “anti-infectives” includes antibacterial, antiviral, antifungal and other anti-pathogen molecules or compounds, that have either cytostatic or cytocidal activities, that act either directly or indirectly by inducing the production of molecules that have a direct cytostatic or cytocidal effect. In one embodiment, the anti-infectives include, but not limited to penicillins and cethylpyridinium chloride.

The term “mucolytic agent” refers any agent that breaks down, or hydrolyzes or liquefies mucus or mucopolysaccharides mucus sufficiently to enhance the antibacterial effect of the monobactam compounds of the composition. Suitable mucolytic agents can include, without limitation, N-acetyl-L-cysteine (MUCOSIL™; Dey Laboratories), recombinant human DNase (PULMOZYME®, Genentech, Inc.), Erdosteine (expectorant that enhances airway secretion and thus reduces viscosity of mucous), Gauifenesin (an approved expectorant), and sodium taurocholate (for in vitro use).

Those knowledgeable in the art will be able to determine the proper effect amount of the mucolytic agent administered to the patient. Examples of dosage ranges of mucolytic agents include Pulmozyme (Domase alpha): 2.5 mg single use ampoule administered once daily by nebulization, Mucofilin (acetylcysteine), 20% solution (200 mg/mL) with 3 to 5 ml of the 20% solution for 3 to 4 times a day administered by nebulization. Expectrorin (guaifenesin): 1-2 tablets administered orally every 12 hours for up to 2400 mg. The mucolytic agent may be administered prior to the monobactam compound and allowed to take effect, such as by waiting a sufficient period of time after administration such as about two to five minutes, or may be administered simultaneously with or after the monobactam compound, as is hereinafter further described.

Anti-inflammatory drugs used in compositions of the present invention can be a steroidal anti-inflammatory agent, a nonsteroidal anti-inflammatory agent, or a combination thereof. In some embodiments, anti-inflammatory drugs include, but are not limited to, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, icloprofen, cintazone, cliprofen, clobetasol propionate, clobetasone butyrate, lopirac, cloticasone propionate, cormethasone acetate, cortodoxone, deflazacort, esonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, alosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, prodrugs thereof, co-drugs thereof, and combinations thereof.

The analgesic herbs containing natural pain reliever used in the compositions of the invention can include, but not limited to, allspice, aloe, arnica, bay laurel, bay rum, bee balm, boneset, boswellia, camphor, caraway, cardamom, cat's claw, chamomile, cinnamon, clove, coriander, devil's claw, eucalyptus, fennel, feverfew, Fo-Ti root, geranium, ginger, hemp/cannabis, hops, immortelle, Jamaican dogwood, lavender, marjoram, nettle, niaouli, nutmeg, palmarosa, peppermint, pine, rehmannia, rosemary, sage, spearmint, pine, tea tree, thyme, turmeric, vetiver, white willow, wintergreen, witch hazel and yucca. Other herbs include, but not limited to, piper methysticum (Kava Kava), echinacea angustifolia, eucalyptus globulus, thymus vulgaris, lycopodium clavatum, phytolacca dencadra, capsicum annum menthe piperita or combination thereof.

In one embodiment, the compositions of the invention comprise an effective amount of hypertonic saline, menthol, piper methysticum (Kava Kava), echinacea angustifolia, eucalyptus globulus, thymus vulgaris, lycopodium clavatum, phytolacca dencadra, capsicum annum mentha piperita and phosphorus.

Another embodiment provides method for treating sore throat or symptoms thereof by orally administering to a patient suffering therefrom a therapeutic composition comprising menthol and at least 1 percent of hypertonic saline, preferably between about 2 to about 30 percent of hypertonic saline, preferably between about 2 to about 20 percent of hypertonic saline, preferably between about 2 to about 15 percent of hypertonic saline, most preferably between about 3 to about 10 percent of hypertonic saline; optionally further comprising buffer agents, surfactants, flavorings, topical oral anesthetics, anti-infectives, mucolytic agents, anti-inflammatory agents, analgesic herbs or combinations thereof.

Another object is to provide a method for the alleviation of discomfort caused by pharyngitis by orally administering to a patient suffering therefrom a therapeutic composition containing from about 3 to about 10 percent of a hypertonic saline and 0.02 to about 5 percent menthol.

In one embodiment, the compositions of the invention are prepared in a total fill volume of at least 1 ml, preferably ranging from about 2 ml to about 500 ml, preferably about 5 ml to about 250 ml, preferably about 10 ml to about 150 ml, most preferably about 15 ml to about 120 ml.

In one embodiment, compositions of the invention can have a pH in the range of about pH 4 to about pH 8, in particularly from about pH 5 to about pH 7.5. The pH may be adjusted by the addition of a solution containing an acidic salt or an acid (e.g. hydrochloric or sulfuric); or of a basic salt or a base (e.g. sodium hydroxide). In case of insufficient stability of the formulation, aqueous buffered systems (citrate, acetate, phosphate) may be added to keep the pH within a physiologically acceptable range. A variety of buffers known in the art may be used in the formulation of the invention, such as various salts of organic or inorganic acids, bases, or amino acids, and including various forms of citrate, phosphate, tartrate, succinate, adipate, maleate, lactate, acetate, bicarbonate, or carbonate ions.

In one embodiment, the composition of the invention can be modified to accommodate a particular patient population such as, but not limited to, cystic fibrosis.

The composition of the present invention can be administered to a human being suffering from the discomfort of pharyngitis in either a liquid or a solid form. Suitable liquid preparations can be prepared wherein the concentration of the hypertonic saline is 0.5 to 20 percent, the preferred concentration being 3.0 to 10 percent and the concentration of menthol is 0.02 to 10 percent, the preferred concentration being 0.04 to 5 percent. The liquid preparations are suitable for use as a spray or they can be sipped in small amounts. Suitable solid forms of the hypertonic saline and menthol can be a pastilles, troches, lozenges, dragees or in any other suitable solid form which can be retained in the mouth while the active ingredient is slowly released by the dissolving of the solid preparation. The concentration of the hypertonic saline and menthol in the solid preparation can be 0.5 to 20 percent, the preferred concentration being 3.0 to 10.0 percent.

While this invention has been particularly shown and described with references to preferred embodiments thereof, it will be understood by those skilled in the art that various changes in form and details may be made therein without departing from the scope of the invention encompassed by the appended claims.

1. A composition for the use in treating sore throat comprising an effective amount of hypertonic saline for osmotic action in the oral-pharyngeal epithelial mucosa and menthol.

2. The composition according to claim 1 wherein the concentration of hypertonic saline is in the range of about 3 to about 10 percent.

3. The composition according to claim 1 wherein the concentration of hypertonic saline is about 3.5 percent.

4. The composition according to claim 1 wherein the concentration of hypertonic saline is about 7 percent.

5. The composition according to claim 1 wherein the concentration of menthol is in the range of about 0.04 to about 5 percent.

6. The composition according to claim 1, further comprising topical oral anesthetics, buffer agents, surfactants, preservatives, flavorings, anti-infectives, mucolytic agents, anti-inflammatory agents, analgesic herbs or combination thereof.

7. The composition of claim 6, wherein the topical oral anesthetics is benzocaine, phenol, dyclonine, Piper methysticum (Kava Kava), or combination thereof.

8. The composition according to claim 1, wherein said composition is in the form of a liquid.

9. The composition according to claim 8, wherein the liquid is being administered as a throat spray.

10. The composition according to claim 1, wherein said composition is in the form of a lozenge.

11. A method for treating sore throat by orally administering to a patient suffering therefrom a therapeutic composition containing hypertonic saline and menthol.

12. The method according to claim 11, wherein the concentration of hypertonic saline is in the range of about 3 to about 10 percent.

13. The method according to claim 11, wherein the concentration of menthol is in the range of about 0.04 to about 5 percent.

14. The method according to claim 11, further comprising topical oral anesthetics, buffer agents, surfactants, preservatives, flavorings, anti-infectives, mucolytic agents, anti-inflammatory agents, analgesic herbs or combination thereof.

15. The method according to claim 14, wherein the topical oral anesthetics is benzocaine, phenol, dyclonine, Piper methysticum (Kava Kava), or combination thereof.

16. The method according to claim 11, wherein said composition is in the form of a liquid.

17. The method according to claim 16, wherein the liquid is being administered as a throat spray.

18. The method according to claim 11, wherein said composition is in the form of a lozenge.
